(19) United States
(12) Patent Application Publication
(10) Pub. No.: US 2013/0150675 A1
(43) Pub. Date: Jun. 13, 2013

(54) Transparent Speculum Apparatus and System

(71) Applicant: Lillian Folley, Altamonte Springs, FL (US)

(72) Inventor: Lillian Folley, Altamonte Springs, FL (US)

(21) Appl. No.: 13/705,620

(22) Filed: Dec. 5, 2012

Related U.S. Application Data
(60) Provisional application No. 61/567,990, filed on Dec. 7, 2011.

(51) Int. Cl. A61B 1/227; A61B 1/233; A61B 1/06

(52) U.S. Cl. CPC A61B 1/227; A61B 1/06; A61B 1/233
USPC 600/200

(57) ABSTRACT

A system for inspecting a body cavity of a patient comprises an otoscope having a gripping body, a speculum having a narrow distal portion adapted to be placed in the body cavity of the patient and a wide proximal portion connectable to a top end of the gripping body, a viewing port connected to the top end of the gripping body to provide a line of sight through the speculum, and a light source connected to the top end of the gripping body configured to direct light through the speculum, where the speculum is substantially transparent.

### REFERENCE TO RELATED APPLICATION

This application claims priority to U.S. Provisional Patent Application No. 61/567,990, filed Dec. 7, 2011, entitled “A transparent (clear) bodied ear speculum.”

### BACKGROUND OF THE INVENTION

The present invention relates to an entirely transparent specula (otoscope attachment) enabling a 360 degree unobstructed view of the walls of the ear canal or nares.

Currently, when using an otoscope, a Health Care provider is only able to view through the viewing port lens on the top end of the gripping body of the Otoscope into the large diameter opening at the end of the speculum and see in a line of sight completely through the ear speculum to the small distal end opening (tip) of the speculum to visualize inside the ear canal. There is a need for an improvement.

### SUMMARY OF THE INVENTION

In one aspect of the present invention, a system for inspecting a body cavity of a patient comprises an otoscope having a gripping body; a speculum having a narrow distal portion adapted to be placed in the body cavity of the patient and a wide proximal portion connectable to a top end of the gripping body; a viewing port connected to the top end of the gripping body to provide a line of sight through the speculum; and a light source connected to the top end of the gripping body configured to direct light through the speculum, wherein the speculum is substantially transparent.

In one aspect, the speculum is entirely transparent. In one aspect, the distal portion of the speculum is substantially cylindrical and the proximal portion of the speculum is substantially frustoconical. In one aspect, the gripping body comprises an elongated tubular body comprising a gripping surface having at least one of indentations and protuberances. In one aspect, the gripping surface is knurled.

In one aspect of the present invention, a method of inspecting a body cavity of a patient comprises: providing the system as described; inserting the distal portion into the body cavity of the patient; illuminating the body cavity of the patent with the light source; inspecting the body cavity of the patient through a line of sight passing through the distal portion of the speculum; and inspecting the body cavity of the patient through a line of sight passing through a transparent region of the proximal portion of the speculum.

In one aspect, the body cavity is an ear canal of the patient. In one aspect, the body cavity is a nostril of the patient. In one aspect, the speculum comprises plastic and the method further comprises disposing of the speculum. In one aspect, the method further comprises reusing the speculum.

These and other features, aspects and advantages of the present invention will become better understood with reference to the following drawings, description and claims.

### BRIEF DESCRIPTION OF THE DRAWINGS

FIG. 1 is a perspective view of the invention shown in use.

FIG. 2 is a perspective view of the invention shown attached to an otoscope device.

FIG. 3 is a perspective view of a transparent speculum according to one embodiment.

FIG. 4 shows a cross sectional view of an ear canal being inspected by a system according to one embodiment.

FIG. 5 is a perspective view of a transparent speculum according to one embodiment.

FIG. 6 is a perspective view of a transparent speculum according to one embodiment.

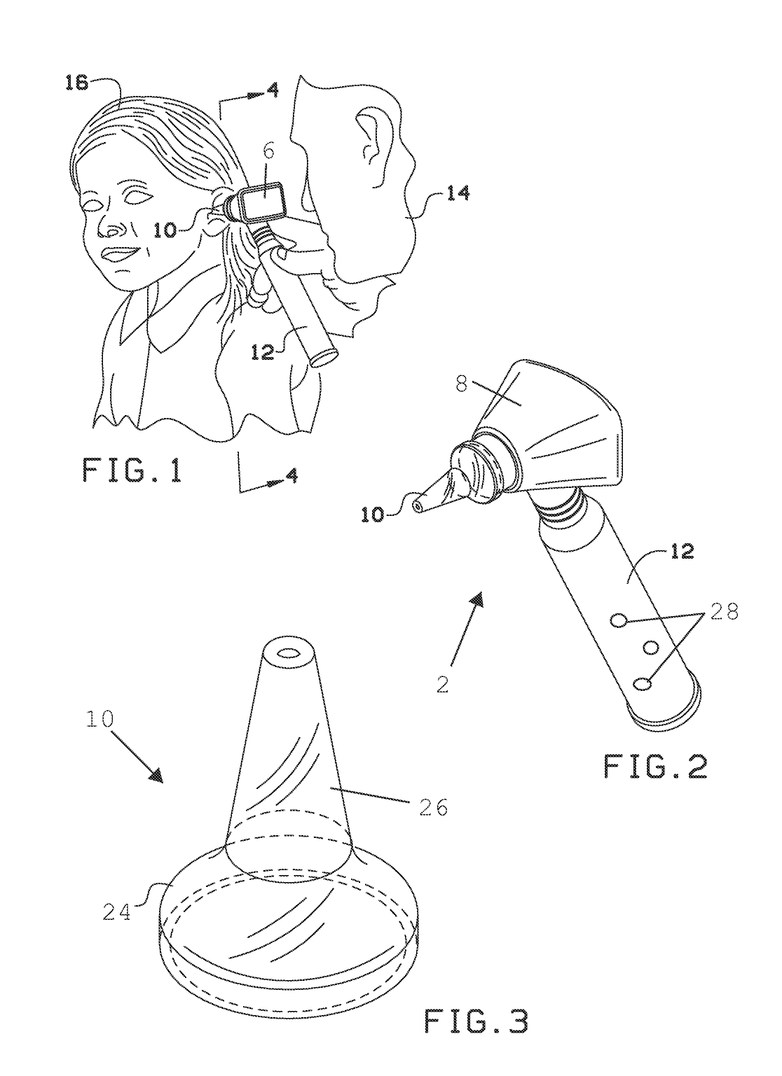

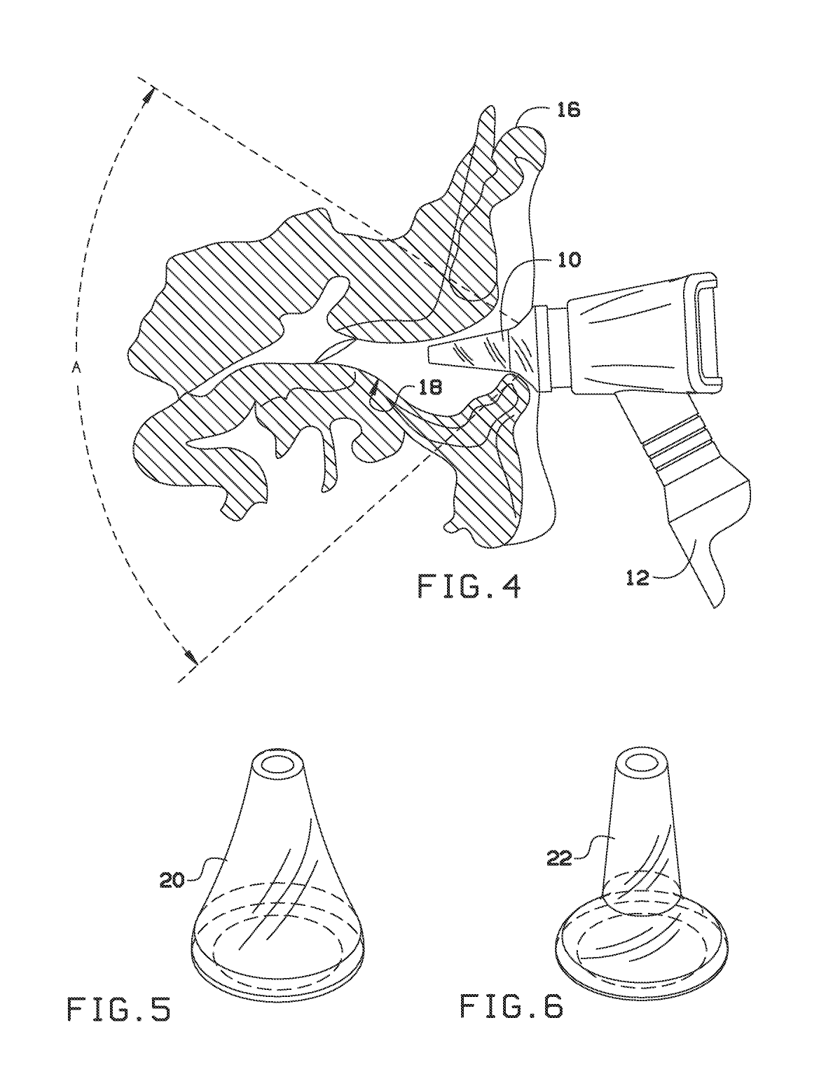

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention.

The present invention is a new variation/design of a disposable ear speculum which is attached to the top end of an Otoscope.

This invention/design is an entirely transparent (clear) speculum made or formed from varied materials or plastics or polycarbonate plastics. The speculum is produced utilizing a completely see through/transparent material for the entire device/form/body of the conical shaped ear speculum (instead of the black plastic or opaque material currently used for the device/form/body of an ear speculum).

An otoscope is a medical device normally designed to allow a Health Care Provider to peer inside an ear or into a nose through a lighted pathway. A variety of illuminated otoscopes are used for examination of the ear. Typically, these otoscopes are devices in which a hollow, generally cone-shaped (or funnel shaped) speculum is attached to the top end of the gripping body of the otoscope. The speculum with an outer or distal end adapted to be placed into the ear of the patient is inserted into the ear canal to enable a view of the outer and middle ear to evaluate the ear during routine examinations or to investigate and diagnose problems with the ear.

The objectives of the present invention/design include:
Utilizing a transparent or clear material for the speculum will allow the examiner to not only see straight through to the tip of the speculum, but will also allow a 360 degree unobstructed view of the walls of the ear canal through the transparent (clear) sides/body of the speculum. This will provide the examiner with a full perspective of the entire ear canal during the exam. (Similar to the view obtained when using a clear plastic speculum during a vaginal exam)
Diseases which may be diagnosed by an Otoscope include, but are not limited to, Otitis media and Otitis Externa, infection of the middle and outer parts of the ear, respectively. The ability to have available and use a transparent (clear) bodied plastic speculum will provide the Health care Provider with a significantly improved diagnostic tool.
It is an objective of the inventor to provide a speculum which enhances the use of the otoscope.

Typically, an ear speculum is a generally cone-shaped device that is inserted into the ear canal. There is, therefore, provided in practice of this invention an improved ear, nose and throat examining instrument. These and other objectives of the invention will become apparent from a review of the appended specifications.

Referring now to the drawings, the present invention includes an otoscope having a gripping body; a hollow ear speculum, having an outer end adapted to be placed in the ear canal of a patient, said ear speculum being connected to a top end of the gripping body; a viewing port connected to the top end of said gripping body to provide a line of sight through the ear speculum; a light source which is directed through said ear speculum; and a transparent (clear) bodied speculum.

The exterior of the otoscope comprises a gripping barrel which is an elongated tubular body, having a knurled surface which facilitates the holding of the otoscope in one hand. Shown is a transparent (clear) body speculum showing the large diameter opening at the end of the speculum which attaches to the otoscope. Shown is a transparent (clear) bodied speculum with the small distal end opening (tip) of the speculum which inserts into the ear canal to visualize inside the ear canal depicted at the upper end of the illustration.

The Transparent (clear) bodied speculum will be sized to engage the ear speculum to an otoscope with a friction fit, which permits removal of the speculum for cleaning, or for mounting a different size speculum, i.e., a pediatric sized speculum. The speculum could be disposable or re-usable. The ear speculum has a generally sloped frustoconical shape as shown and a smooth surface that permits insertion of the ear speculum into the ear canal to a limited depth without injuring the ear.

A system for inspecting a body cavity of a patient 16 comprises: an otoscope 2 having a gripping body 12; a speculum 10 having a narrow distal portion 26 adapted to be placed in the body cavity of the patient 16 and a wide proximal portion 24 connectable to a top end of the gripping body 12; a viewing port 6 connected to the top end of the gripping body 12 to provide a line of sight through the speculum 10; and a light source 8 connected to the top end of the gripping body 12 configured to direct light through the speculum 10, wherein the speculum 10 is substantially or entirely transparent.

The distal portion 26 of the speculum 10 may be frustoconical or substantially cylindrical and the proximal portion 24 of the speculum 10 may be substantially frustoconical. The gripping body 12 may comprise an elongated tubular body comprising a gripping surface having at least one of indentations 28 and protuberances 28. The gripping surface 12 may be knurled. FIGS. 5 and 6 show alternative embodiments of the speculum 20, 22.

The foregoing description of a preferred embodiment of the invention has been presented for purposes of illustration and description. It is not intended to be exhaustive or to limit the invention to the precise form disclosed. Obvious modifications or variations are possible in light of the above teachings and fitting to existing otoscopes and insertion into ear canals. All such obvious modifications and variations are intended to be within the scope of the invention.

What is claimed is:

1. A system for inspecting a body cavity of a patient, comprising:
an otoscope having a gripping body;
a speculum having a narrow distal portion adapted to be placed in the body cavity of the patient and a wide proximal portion connectable to a top end of the gripping body;
a viewing port connected to the top end of the gripping body to provide a line of sight through the speculum; and
a light source connected to the top end of the gripping body configured to direct light through the speculum,
wherein the speculum is substantially transparent.

2. The system as claimed in claim 1, wherein the speculum is entirely transparent.

3. The system as claimed in claim 1, wherein the distal portion of the speculum is substantially cylindrical and the proximal portion of the speculum is substantially frustoconical.

4. The system as claimed in claim 1, wherein the gripping body comprises an elongated tubular body comprising a gripping surface having at least one of indentations and protuberances.

5. The system as claimed in claim 4, wherein the gripping surface is knurled.

6. A method of inspecting a body cavity of a patient, comprising:
providing the system as claimed in claim 1;
inserting the distal portion into the body cavity of the patient;
illuminating the body cavity of the patent with the light source;
inspecting the body cavity of the patient through a line of sight passing through the distal portion of the speculum; and
inspecting the body cavity of the patient through a line of sight passing through a transparent region of the proximal portion of the speculum.

7. The system as claimed in claim 6, wherein the body cavity is an ear canal of the patient.

8. The system as claimed in claim 6, wherein the body cavity is a nostril of the patient.

9. The system as claimed in claim 6, wherein the speculum comprises plastic and the method further comprises disposing of the speculum.

10. The system as claimed in claim 6, wherein the method further comprises reusing the speculum.
